(19) United States
(12) Patent Application Publication
(10) Pub. No.: US 2009/0196395 A1
(43) Pub. Date: Aug. 6, 2009

(54) CEPHALOMETRIC X-RAY IMAGING APPARATUS

(75) Inventors: John Joseph GREGORIO, Chana, IL (US); Omid Ebrahimi KIA, North Bethesda, MD (US); Krishnamoorthy SUBRAMANYAN, Palatine, IL (US)

Correspondence Address: MCNEES WALLACE & NURICK LLC, 100 PINE STREET, P.O. BOX 1166, HARRISBURG, PA, 17108-1166, US

(73) Assignee: GENDEX CORPORATION, Washington, DC (US)

(21) Appl. No.: 12/388,344

(22) Filed: Feb. 18, 2009

Related U.S. Application Data
(60) Provisional application No. 61/074,564, filed on Jun. 20, 2008.

(51) Int. Cl. A61B 6/14

(52) U.S. Cl. 378/38

(57) ABSTRACT

A dental x-ray diagnostic apparatus and method for performing cephalography is disclosed that rotates an x-ray source generating an x-ray beam while synchronously linearly moving the secondary collimator and x-ray imager to produce the cephalometric image. The x-ray beam is swept through a position where a detector is positioned for performing panoramic scanning.

### CROSS-REFERENCE TO RELATED APPLICATIONS

This application claims priority to U.S. Provisional Patent Application No. 61/074,564 filed on Jun. 20, 2008, entitled “CEPHALOMETRIC X-RAY IMAGING APPARATUS” which is herein incorporated by reference in its entirety.

### FIELD

This invention relates to an apparatus and method for performing cephalometric x-ray imaging or scanning. The invention further relates to an apparatus and method for rotating an x-ray source about a vertical axis through the focal point of the x-ray source to perform a traversing sweep of an x-ray beam during a cephalometric imaging operation.

### BACKGROUND

Orthopantomography, Scannography, Linear Tomography and Cephalography are complementary dental imaging techniques, often incorporated into a single piece of equipment that can be used to obtain a comprehensive survey of the maxillo-facial complex, tomographic views of selected anatomical districts under transversal or axial projections, and cranial views under multiple projections.

Orthopantomography aims to produce an image of a curved plane approximating the patient jaw, with blurring of the anatomical structures laying outside of a narrow layer around the curved plane, by using the relative movement of film or a sensor versus the rotation of an x-ray source to generate a layer forming effect.

Scannography has a layer forming process similar to Orthopantomography, where the object is typically laying on a flat plane. It is practically used to produce axial or transverse views of specific anatomical districts, such as the jaw, the joints and the sinus.

Linear Tomography uses classic linear tomographic layering to produce axial or transverse views of specific anatomical districts in the jaw.

Cephalography is an imaging technique, which produces radiographic images of the cranial complex under various projections, with minimum magnification and geometrical distortion.

For all of the above imaging modalities, real-time, digital x-ray image acquisition is preferable to the use of film because digital imaging eliminates film processing and related chemicals and takes advantage of the improved performances and reduced costs provided by modern image sensor technology.

U.S. Pat. No. 4,188,537 describes apparatus and methods in which Realtime Digital Panoramic Radiography is implemented by an array of multiple detectors, or a vertical scanning single detector, where vertical lines are acquired in synchronization with the rotation movement to generate and display a panoramic image.

U.S. Pat. No. 4,878,234 describes apparatus and methods in which Real-time Digital Panoramic Radiography is implemented by CCD image sensors where vertical lines in the image zone are clocked out in the not-illuminated storage zone by a frequency simulating the speed of the moving x-ray film.

In U.S. Pat. No. 4,823,369, Real-time Digital Panoramic Radiography is implemented by x-ray image detectors, preferably consisting of amorphous silicon, where complete frames corresponding to the active area are acquired at sufficiently fast frequency and adjacent frames are added as a function of time, either by pre-processing in order to obtain the panoramic image on one selected layer, or by storing in memory and later processing, so giving the possibility of multiple layer reconstruction.

U.S. Pat. No. 4,995,062 describes an apparatus and methods in which Real-time Digital Panoramic Radiography is implemented by CCD image sensors where different vertical lines are driven with different clock frequencies for simultaneously obtaining a plurality of tomograms at different depths of the jaw.

U.S. Pat. No. 5,195,114 describes apparatus and methods in which Real-time Digital Panoramic Radiography is accomplished by an x-ray image detection system, typically based on a signal intensifier tube camera (SIT), where the video signal is acquired and stored in a storage unit (such as video tape recorder). Frame digital data derived by A/D conversion and processed selecting frame interval and shift depending on the movement speed of the target, are used to digitally form the panoramic image of given tomographic layers. This arrangement is limited in the video rate acquisition, and does not provide enough resolution for adequate panoramic image reconstruction. The process is also time consuming and, in case of digital frame storage, would require huge amount of memory.

European Patent 0 673 623 describes apparatus and methods in which Real-time Digital Panoramic Radiography is implemented by an x-ray detection system having an area coincident with the cross-section area of the x-ray and requiring only one narrow slit x-ray diaphragm located on the x-ray source. By this arrangement, the panoramic image reconstruction is accomplished either by frame acquisition, with intermediate frame storage (memory consuming option) or with immediate frame processing (less memory consuming option), or by the TDI method. In the first case, for adequate layer formation, the frame resolution must be chosen in a way to ensure that each point of the final reconstructed image is represented in more positionally shifted images (preferably five or more). In the second case (even less memory consuming), the image is directly integrated and formed on the x-ray detector by controlling the clock sequence in a way to ensure that the projected image of a point within the sharp layer of the object will be represented by the same spatial position in the final reconstructed panoramic image.

In previous cephalometric systems, a “C”-arm can rotate and move relative to the scanned object to maintain the proper alignment of the x-ray beam and x-ray detector. However, the combination of rotational and linear movements of the C arm increases the complexity of the system, increasing system, operation and maintenance costs.

### SUMMARY

The present invention provides an x-ray apparatus and method capable of performing an improved cephalometric scanning modality.

In accordance with an embodiment of the invention, a system for performing a cephalometric scan is disclosed that includes a kinematic assembly attached to a frame, a rotary unit attached to the kinematic assembly, an x-ray source rotatably connected to the rotational assembly about a first rotational axis, a detector located along a first axis at a first distance from the x-ray source, a secondary collimator disposed along the first axis between the primary collimator and the detector, and a control system configured to rotate the x-ray source about the first rotational axis over a controlled sweep angle while synchronously moving the detector and the secondary collimator in a direction along a second axis perpendicular to the first axis.

In accordance with another embodiment of the invention, a method for performing a cephalometric scan is disclosed that includes positioning an x-ray source and a detector at a first distance along a first axis, generating an x-ray beam from the x-ray source, rotating the x-ray source about a fixed first rotational axis on the first axis while sweeping the x-ray beam over a controlled sweep angle, and synchronously moving a secondary collimator and the detector in a direction along an axis approximately perpendicular to the first axis while the detector receives the generated x-ray beam to perform a cephalometric scan.

Other features and advantages of the present invention will be apparent from the following more detailed description of the preferred embodiments, taken in conjunction with the accompanying drawings which illustrate, by way of example, the principles of the invention. The scope of the invention is not, however, limited to these preferred embodiments.

### BRIEF DESCRIPTION OF THE DRAWINGS

FIG. 1 is an illustration showing a prior dental x-ray diagnostic system.

FIG. 2 is an illustration showing an exemplary dental x-ray diagnostic system according to the present invention.

FIG. 3 is a schematic diagram of an exemplary control system for the present invention.

FIG. 4 is a schematic diagram illustrating an example of the relative movement of the dental x-ray diagnostic system from a top perspective.

Wherever possible, the same reference numbers will be used throughout the drawings to refer to the same or like parts.

### DETAILED DESCRIPTION

The present invention provides a system and method for performing an improved cephalometric scanning modality. The improved method includes rotating the x-ray source of an x-ray diagnostic apparatus while keeping the “C”-arm stationary. The improved x-ray diagnostic apparatus is configured to prevent the rotation and traversing of the “C”-arm during the cephalometric scanning while rotating only the x-ray source to obtain a cephalometric scan.

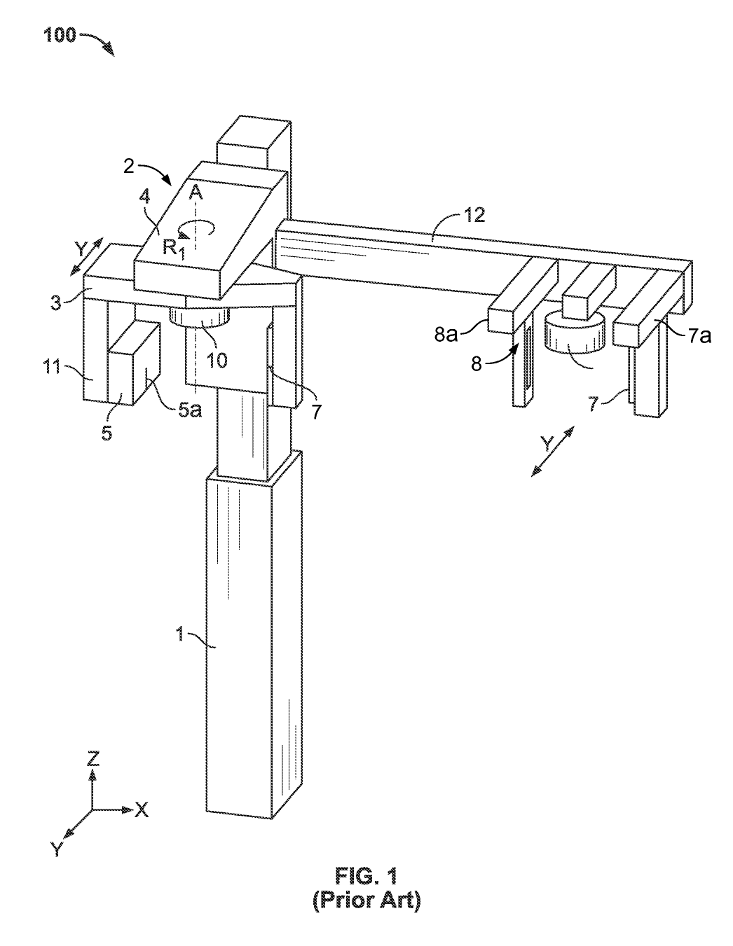

Referring to FIG. 1, an exemplary prior art dental x-ray diagnostic system 100 for performing panoramic and cephalometric scanning modalities is provided. A dental x-ray diagnostic system 100 is discussed in U.S. Pat. No. 7,197,107, granted Mar. 27, 2007, which is incorporated by reference in the entirety, herein. The system 100 includes a base 1 and a frame 2 that slides vertically along the base 1. The configuration of the base 1 and frame 2 provides for a vertical height adjustment of the system 10 during patient positioning, and may also be used for vertical scanning in a cephalometric arrangement of the system 10. The frame 2 is provided with an independent actuator (not shown) for the control of the vertical movement of the frame 2.

The system 100 further includes a rotary unit 3 connected to the frame 2 by a support such as kinematic assembly 4. The rotary unit 3 or kinematic assembly 4 is provided with actuators (not shown) that provide X linear movement, Y linear movement and R₁ rotational movement of the rotary unit 3 about the point of attachment to the kinematic assembly 4 to perform both panoramic and cephalometic scanning. As used herein, the term “actuator” includes, but is not limited to, any combination of actuators, motors and drives to provide the stated movement. The rotary unit 3 supports an x-ray source 11 fixed at one end, and a detachable x-ray imager 7 at an opposite end. The x-ray imager 7 positioned at the opposite end of the rotary unit 3 can be used for panoramic scanning or other imaging modalities.

The x-ray source 11 generates an x-ray beam having a focal spot. A primary collimator 5 having an aperture 5a for controlling the x-ray beam width and height is affixed to the x-ray source 11. The primary collimator 5 may include sliding slabs (not shown) configured to adjust the opening height and width of the aperture 5a to control the x-ray beam width and height to provide dynamic collimation.

The system 100 further includes an arm 12, which is affixed at one end to frame 2. An x-ray imager 7 is attached to an opposite end of the arm 12 by an actuator 7a. The x-ray imager 7 attached at the end of arm 12 can be used for cephalometric image acquisition. The actuator 7a provides Y linear movement to the x-ray imager 7. In one embodiment, the x-ray imager 7 attached at the end of arm 12 may be the same x-ray imager 7 attached in the panoramic image acquisition position at the end of rotary unit 3 repositioned to the end of arm 12. In another embodiment, the x-ray imager 7 in the panoramic position and the x-ray imager 7 at the end of arm 12 in the cephalometric position may be separate imagers. The arm 12 may provide for telescoping, swinging, folding, or other horizontal and/or vertical movement to position the x-ray imager 7 for panoramic, cephalometric or other modality.

A secondary collimator 8 is attached to arm 12 by an actuator 8a. Secondary collimator 8 is disposed between x-ray source 11 and x-ray imager 7 attached in the ceph position. Actuator 8a provides Y linear movement to the secondary collimator 8. Actuator 8a may be independently controlled or controlled in conjunction with actuator 7a.

A first patient positioning system 10 is attached to the frame 2 proximate the rotary unit 3 for panoramic, scannographic, and linear tomography modalities. A second patient positioning system 9 is attached to arm 12 for cephalometric modalities. In alternative embodiments, either one or both of the first and second patient positioning systems 10, 9 may be attached to the base 1 or to a floor or wall surface (not shown). One or both of the first and second patient positioning systems 10, 9 may be provided with an actuator to provide for adjustments to patient height.

To perform a cephalometric scan with the dental x-ray diagnostic system 100, a patient is positioned by the second patient positioning system 9. The x-ray imager 7 is positioned in the cephalometric position at the end of arm 12, if not already in that position. The x-ray source 11 may be linearly moved in the Y direction, linearly moved in the X direction, rotated R₁ about the A axis, or any combination thereof, while synchronously linearly moving the secondary collimator 8 and the x-ray imager 7 in the Y direction to produce a cephalometric image.

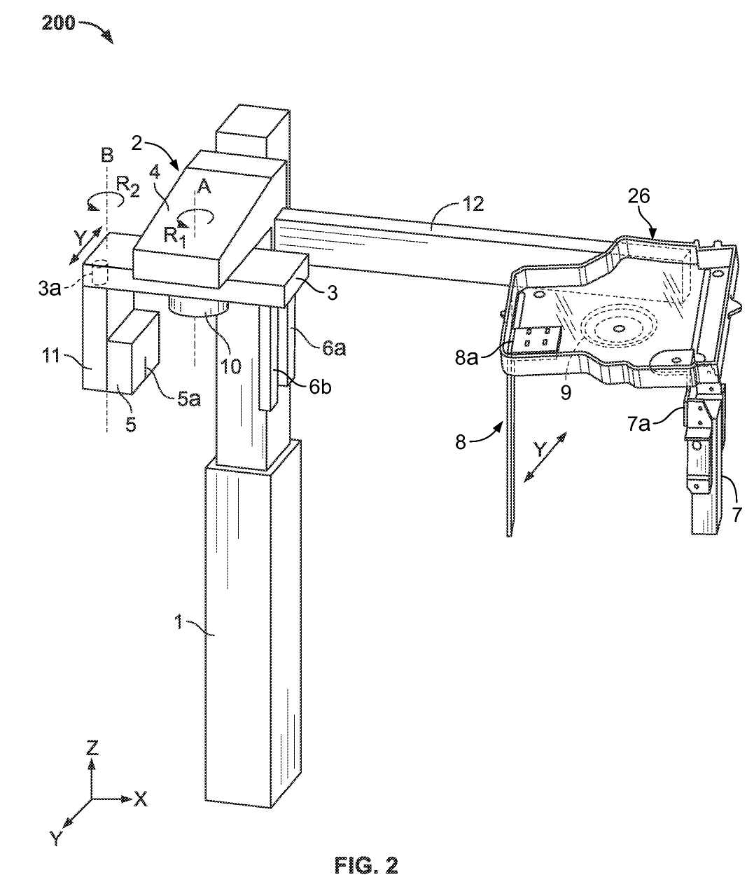

FIG. 2 shows an exemplary embodiment of a dental x-ray diagnostic system (system) 200 for performing cephalometric scanning according to the invention. The system 200 includes a base 1 and a frame 2. In one embodiment, the system 200 can be configured to provide vertical movement in the Z direction to the frame 2, the first positioning system 10, the second positioning system 9, and any combination thereof. Vertical movement of the frame 2 may be used for patient positioning, and may also be used for vertical scanning in a cephalometric arrangement of the system 200. In another embodiment, the base 1 and frame 2 may be configured without providing vertical movement. For example, the base 1 and frame 2 may be combined into a single component that does not provide for vertical movement.

The system 200 further includes a kinematic assembly 4 located at one end of frame 2. A rotary unit 3 is connected to frame 2 by a support such as kinematic assembly 4. The system 200 can further be configured to provide horizontal linear movement in the X-direction to the kinematic assembly 4, the rotary unit 3, the arm 12, and any combination thereof. The system 200 can further be configured to provide rotation movement R₁ about an A axis to the rotary unit 3.

An x-ray source 11 is rotatably attached at one end of the rotary unit 3 by a coupling 3a. The system 200 is configured to provide the x-ray source rotation movement R₂ about a B axis as shown in FIG. 2. In one embodiment, the system 200 is configured to rotate the x-ray source 11 about the B axis up to at least approximately 12 degrees. In another embodiment, system 200 is configured to rotate the x-ray source about the B axis up to at least approximately 5 degrees. In yet another embodiment, system 200 is configured to rotate the x-ray source about the B axis by up to about 2 degrees.

The x-ray source 11 includes an output port (not shown) proximate a primary collimator 5. In this exemplary embodiment, the primary collimator 5 is attached to the x-ray source 11. The primary collimator 5 provides a controlled beam width and height to the x-ray beam generated by the x-ray source 11. The primary collimator 5 includes an aperture 5a for controlling the x-ray beam width and height. The primary collimator 5 may include sliding slabs (not shown) configured to adjust the opening height and width of the aperture 5a to control the x-ray beam width and height to provide dynamic collimation.

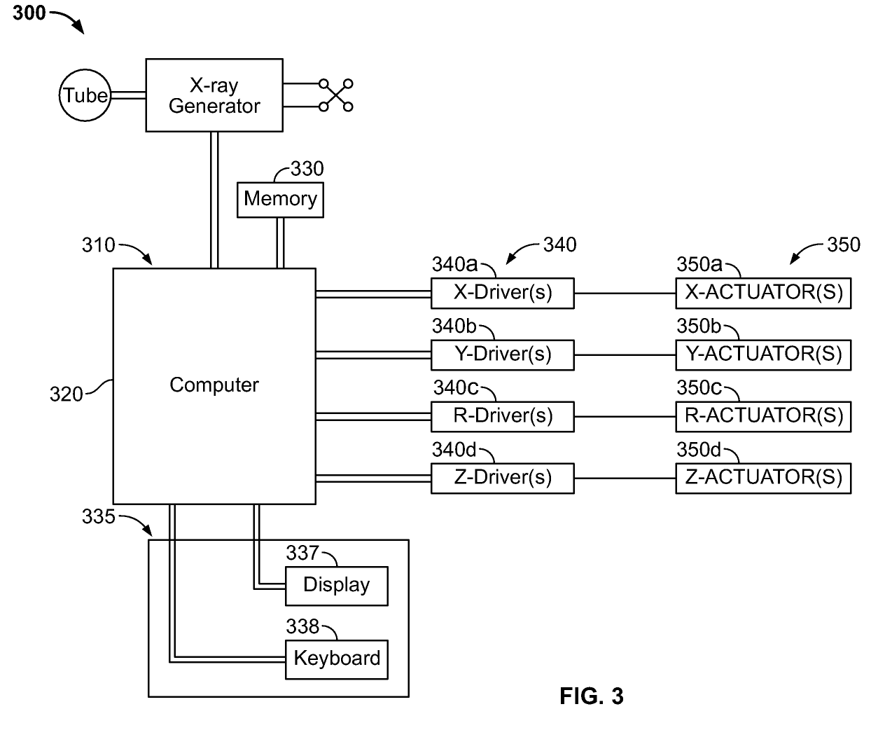

The system 200 further includes an attachment structure 6 attached to an end of rotary unit 3 opposite the end supporting x-ray source 11. The attachment structure 6 includes arms 6a, 6b. The arms 6a, 6b are separated by a minimum distance dₘᵢₙ (FIG. 3). The attachment structure 6 provides for attachment of x-ray imager 7 for imaging in a panoramic and/or other scanning operation. As can be seen in FIG. 2, the x-ray imager 7 is removed to configure the system 200 to perform a cephalometric scan according to the invention. In one embodiment, attachment structure 6 is integral to the rotary unit 3. In another embodiment, the attachment structure 6 is a separate and/or removable component from the rotary unit 3.

FIG. 2 further shows a cephalometric detector platform (platform) 26 positioned at an end of arm 12 opposite the source 11 in a cephalometric scanning position. The platform 26 includes an x-ray imager 7 and a secondary collimator 8. The x-ray imager 7 is attached to the platform 26 by an independent active actuator 7a configured to provide linear Y movement to the x-ray imager 7. In another embodiment, the actuator 7a is included in the platform 26. In one embodiment, the x-ray imager 7 is positioned at a predetermined distance of approximately 150 cm from the x-ray source 11 for performing cephalometric scanning. In an alternative embodiment, the arm 12 and/or platform 26 may be configured to provide for telescoping, swinging, folding, or other horizontal and/or vertical movement to position the x-ray imager 7. In yet another embodiment, the arm 12 and/or platform 26 are configured to provide X direction and Y direction movement to maintain the x-ray imager 7 at an approximately constant predetermined distance form the x-ray source 11 during cephalometric scanning, or in other words, as the x-ray source 11 rotates about the B axis and the secondary collimator 8 and x-ray imager 7 linearly move in the Y direction.

The secondary collimator 8 is attached to the platform 26 by an actuator 8a configured to provide linear Y movement to the secondary collimator 8. In one embodiment, the actuator 8a is an independent active actuator. In another embodiment, the actuator 8a is included in the platform 26. The secondary collimator 8 is disposed between the x-ray source 11 and the x-ray imager 7. The secondary collimator 8 and the x-ray imager 7 are configured to move in a linear Y movement synchronously with the rotational movement R₂ of the x-ray source 11 about the B axis to perform a cephalometric scan of a patient disposed between the x-ray source 11 and the x-ray imager 7.

A second patient positioning system 9 is attached to the ceph platform 26 for cephalometric modalities. In alternative embodiments, the second patient positioning system 9 may be attached to the base 1, arm 12, or to a floor or wall surface (not shown). The second patient positioning system 9 may be provided with an independent actuator (not shown) for adjustments to patient height.

FIG. 3 shows an exemplary scheme for a control system 300 for the system 200 according to the present invention. The control system 300 includes a controller 310 that includes a computer 320 and associated memory 330. The controller 310 is connected to an operator interface 335 that includes a display 337 and an input device 338. The input device 337 may include a keyboard.

The controller 310 provides independent drivers 340 with cinematic profiles data associated to the specific orbital projection required during the normal operation and during the various scanning processes including the various scanning processes foreseen in cephalography. In another embodiment, the controller may provide profiles data to more or less drivers 340, as the drivers 340 and actuators 350 may be combined or separated into more or fewer independent drivers 340 and actuators 350.

The independent drivers 340 may include X-driver(s) 340a, Y-driver(s) 340b, R-driver(s) 340c and Z-driver(s) 340d that provide instructions to actuators 350. In one embodiment, the control system 300 may include X-driver(s) 340 to provide instruction to X-actuator(s) 350a to provide X linear movement to various system 200 components including, but not limited to the kinematic assembly 4, rotary unit 3, and arm 12 in the X direction. The control system 300 can further include Y-driver(s) 340b to provide instruction to Y-actuator(s) 350b to provide Y linear movement to various system 200 components including, but not limited to the kinematic assembly 4, secondary collimator 8 and x-ray imager 7 positioned for cephalometric scanning. For example, the Y-driver(s) 340b may provide instructions to actuator 8a and actuator 7a to provide Y movement to the primary collimator and x-ray imager 7 positioned for cephalometric scanning, respectively.

In one embodiment, the actuator 8a may be cooperatively controlled with the actuator 7a of the x-ray imager 7. The secondary collimator 8 and the x-ray imager 7 are configured to move in a linear Y movement synchronously with the rotational movement R₂ of the x-ray source about the B axis to perform a cephalometric scan of a patient disposed between the x-ray source 11 and the x-ray imager 7.

The control system 300 can further include R-driver(s) 340c to provide instruction to R-actuator(s) 350c to provide rotational movement to various system 200 components including, but not limited to the rotary unit 3, the x-ray source 11 and the platform 26. The R-actuator(s) can provide rotational movement R₁ of the rotary unit 3 about the A axis or point of attachment to the kinematic assembly 4 to perform panoramic scanning. The R-actuator(s) can also provide rotational movement R₂ of the x-ray source 11 about the B axis or point of attachment to the rotary unit 3 to perform cephalometric scanning.

The control system 300 can further include Z-driver(s) 340d to provide instruction to Z-actuator(s) 350d to provide vertical movement to various system 200 components including, but not limited to frame 2, first positioning system 10, and second positioning system 9 to perform various scanning modalities.

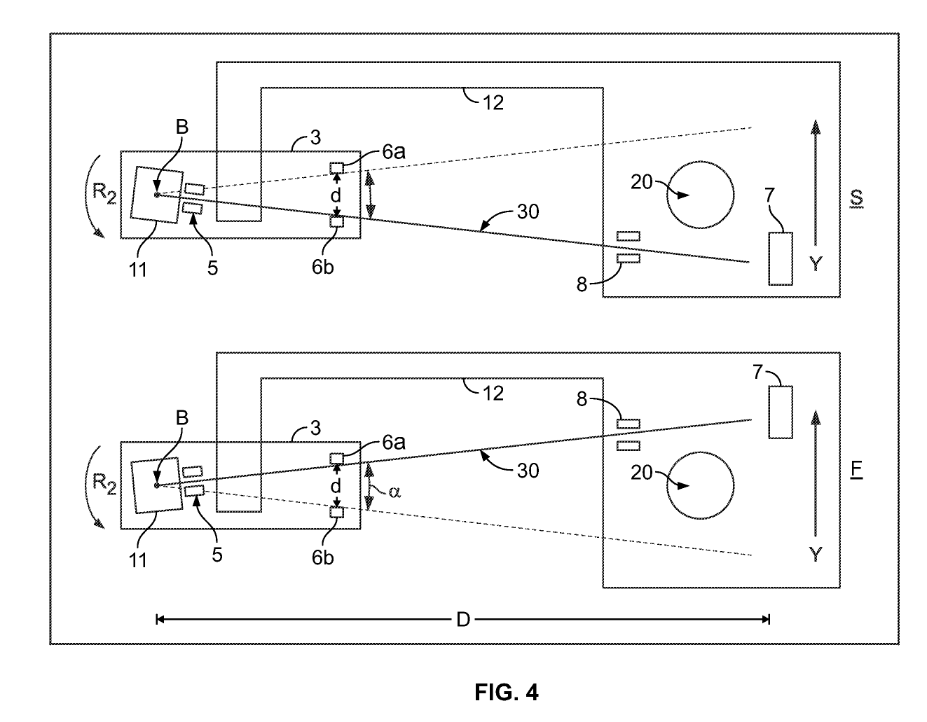

Referring to FIG. 4, a exemplary cephalometric scan of a patient 20 is performed according to the invention by traversing an x-ray beam 30 through an angle α from a start position S to a final position F, while synchronously linearly moving x-ray imager 7 and secondary collimator 8 in the Y direction between start position S to final position F. The rotation of the x-ray source 11 and the movement of the x-ray imager 7 and secondary collimator 8 may be independent, but synchronous. The rotary unit 3 is stationary during the scan. In this exemplary example, angle α is approximately 11.4 degrees. The x-ray beam 30 is traversed through the angle α by rotating x-ray source 11 in a direction R₂ about axis B. The system 300 is configured to limit the rotation R₂ of x-ray source 11 to angle α. As can be seen in FIG. 4, the x-ray beam 30 is traversed between arms 6a, 6b. Arms 6a, 6b are separated by a distance d. In this exemplary embodiment, the distance d is approximately 11 cm.

The x-ray source 11 and the x-ray detector 7 in the cephalometric position are separated by a distance D at the mid-point of the scan through the patient 20. In one embodiment, this distance D may vary slightly during the cephalometric scan. In another embodiment, the distance D may be held approximately constant during the cephalometric scan by adjusting the position of the x-ray source 11 and/or x-ray detector 7. For example, the distance D may be held approximately constant during the cephalometric scan

In another embodiment, arms 6a, 6b are removable from the rotary unit 3. In this another embodiment, the rotation R2 of the x-ray source 11 is limited to angle α. In yet another embodiment, arms 6a, 6b are rotatable or otherwise repositionable to move the support fork arms 6a, 6b (and a panoramic imager 7, if attached) from between the x-ray source 11 and the x-ray imager 7 positioned for cephalometric scanning, and the rotation R2 of the x-ray source 11 is limited to angle α. In still yet another embodiment, the system 200 does not include support fork arms 6 and/or an x-ray imager in the panoramic position, and the rotation R2 of the x-ray source 11 is limited to angle α.

The scan angle α may be controlled to sweep or scan the distance d, or may be controlled to sweep or scan a desired segment of patient 20. In one embodiment, the scan angle α is approximately 11.4 degrees to perform a cephalometric scan having an approximate arc distance of 33 cm at an x-ray detector 7 located in the cephalometric position at a distance of approximately 150 cm from the x-ray source 11.

The control system may be configured to operate the coordinated movement of the x-ray source 11, the secondary collimator 8 and the detector 7, as well as other secondary operations of the system 200. The system 200 may also be configured to perform additional scanning techniques such as tran-scan or other scanning modalities that include synchronous rotation of the x-ray source 11 and rotary unit 3, and with synchronous linear movement of the secondary collimator 8 and x-ray detector 7.

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

1. A system for performing a cephalometric scan, comprising:
a kinematic assembly attached to a frame;
a rotary unit attached to the kinematic assembly;
an x-ray source rotatably connected to the rotational assembly about a first rotational axis;
a detector located along a first axis at a first distance from the x-ray source, a secondary collimator disposed along the first axis between the primary collimator and the detector; and
a control system configured to rotate the x-ray source about the first rotational axis over a controlled sweep angle while synchronously moving the detector and the secondary collimator in a direction along a second axis perpendicular to the first axis.

2. The system of claim 1, wherein the controlled sweep angle is limited to up to about 12 degrees.

3. The system of claim 1, wherein the control system is further configured to maintain the first distance is approximately constant while synchronous moving the detector and the secondary collimator in a direction along a second axis perpendicular to the first axis

4. The system of claim 1, further comprising
a support fork disposed between the x-ray source and the detector;
wherein the support fork is configured to support a detector in a panoramic modality.

5. The system of claim 2, wherein the support fork comprises arms disposed on opposing sides of the first axis.

6. The system of claim 5, wherein control system is further configured to sweep the first axis between the arms.

7. The system of claim 1, wherein the rotary unit is configured to remain stationary during the rotation of the rotary source.

8. A method for performing a cephalometric scan, comprising:
positioning an x-ray source and a detector at a first distance along a first axis;
generating an x-ray beam from the x-ray source;
rotating the x-ray source about a fixed first rotational axis on the first axis while sweeping the x-ray beam over a controlled sweep angle; and
synchronously moving a secondary collimator and the detector in a direction along an axis approximately perpendicular to the first axis while the detector receives the generated x-ray beam to perform a cephalometric scan.

9. The method of claim 8, wherein the controlled sweep angle is limited to up to about 12 degrees.

10. The method of claim 8, wherein the x-ray beam is projected between arms of a support configured to position a detector in a panoramic scanning position.

11. The method of claim 8, wherein the first distance is approximately constant while synchronous while performing the cephalometric scan.

12. The method of claim 8, further comprising:
repositioning a detector disposed in a panoramic scanning position prior to rotating the x-ray source.

13. The method of claim 12, wherein the x-ray beam is swept through detector panoramic scanning.

14. The method of claim 8, further comprising:
repositioning a support configured to support a detector in a panoramic scanning position prior to rotating the x-ray source.
